(19) United States
(12) Patent Application Publication
(10) Pub. No.: US 2024/0424037 A1
(43) Pub. Date: Dec. 26, 2024

(54) Strain of Lactobacillus Pentosus Capable of Relieving Pathological Characteristics of Mice Infected with Influenza Virus and Application Thereof

(71) Applicant: Jiangnan University, Wuxi (CN)

(72) Inventors: Wenwei Lu, Wuxi (CN); Qianwen Wang, Wuxi (CN); Jianxin Zhao, Wuxi (CN); Hongchao Wang, Wuxi (CN); Shenyan Ma, Wuxi (CN); Wei Chen, Wuxi (CN); Hao Zhang, Wuxi (CN)

(21) Appl. No.: 18/829,729

(22) Filed: Sep. 10, 2024

Related U.S. Application Data
(63) Continuation of application No. PCT/CN2023/083004, filed on Mar. 22, 2023.

(30) Foreign Application Priority Data
Apr. 1, 2022 (CN) 202210347835.9

(51) Int. Cl. A61K 35/747; C12N 1/20; C12R 1/225

(52) U.S. Cl. CPC A61K 35/747; C12N 1/205; C12R 2001/225

(57) ABSTRACT

The present disclosure discloses a strain of Lactobacillus pentosus capable of relieving pathological characteristics of mice infected with influenza virus and application thereof, and belongs to the field of microorganisms. The strain of L. pentosus CCFM1227 provided by the present disclosure can effectively improve the innate immune response of the mice infected with influenza virus so as to prevent and/or relieve influenza virus infection. In addition, the L. pentosus CCFM1227 of the present disclosure can be used for fermenting phloretin to produce deaminotyrosine. Therefore, the L. pentosus CCFM1227 has a great application prospect in preparation of products for preventing and/or relieving influenza virus infection.

### REFERENCE TO SEQUENCE LISTING

The instant application contains a Sequence Listing in XML format as a file named “3050-YGHY-2023-45-SEQ.xml”, created on Sep. 9, 2024, of 4771 bytes in size, and which is hereby incorporated by reference in its entirety.

### TECHNICAL FIELD

The present disclosure relates to a strain of Lactobacillus pentosus capable of relieving pathological characteristics of mice infected with influenza virus and application thereof, and belongs to the field of microorganisms.

### BACKGROUND

Flu, the abbreviation of influenza, is different from common cold. The pathogen of the influenza includes influenza viruses. The influenza viruses belong to the genus of influenza viruses in the orthomyxoviridae family, and usually include three types, such as type A, type B and type C. Influenza A virus can infect many hosts, including humans, pigs, birds, whales and other mammals. The influenza has the typical symptoms of fever, dizziness, headache, myalgia, fatigue, anorexia and the like, accompanied by respiratory discomfort, such as nasal congestion, runny nose, cough and the like, and accompanied by potentially fatal complications in severe cases. However, during an influenza pandemic, all infected persons have severe respiratory diseases. The incubation period is 1-5 days, and the symptoms are usually not obvious in the first 3 days.

Four severe influenza pandemics happened in just over 100 years from the end of the 19th century to the beginning of the 21st century, and H1N1 and H3N2 are still pandemic influenza strains nowadays. At present, means for clinical prevention and treatment of the influenza mainly include influenza vaccines and oseltamivir. Influenza virus inactivated vaccines are only registered influenza vaccines used for humans, and current vaccines are triple inactivated vaccines developed based on influenza A viruses H1N1 and H3N2 and influenza B virus. The effectiveness of the influenza vaccines depends on the matching degree with prevalent strains and the composition of the vaccines. The vaccines are used for preventing the influenza based on an immune memory function of adaptive immunity of the body, which are highly specific and only achieve a prevention effect on infected viruses. For mutated influenza viruses, the current vaccines cannot achieve an effective prevention effect. However, the biggest characteristic of the influenza viruses is high variability, and the influenza pandemics happened in the history are also caused by new mutated viruses with high infectivity. According to surveys, the influenza A virus has a big mutation about every ten years. With continuous changes of subtypes, the currently known influenza A virus has a total of 135 subtypes, and the influenza vaccines have certain defects in prevention of the influenza viruses.

In recent years, with progress of research on “lung-gut axis”, intestinal microecology has an obvious correlation with lung diseases. Influenza virus infection may lead to disorder of the intestinal microecology, and on the contrary, intestinal flora and related metabolites may play a key role in resisting the influenza viruses in the body. Due to an immune escape mechanism of the influenza viruses, the virus invasion response of the body is reduced. Therefore, improvement of the immune response of the body is conducive to virus clearance. Studies have shown that intestinal microorganisms help to improve the immunity of the body, enhance the innate immune response during virus invasion, and increase the expression of anti-inflammatory factors to balance the occurrence of inflammation.

It is proven that deaminotyrosine, as a metabolite produced by intestinal bacteria, can activate the type I interferon response in innate immunity to relieve influenza virus infection. The deaminotyrosine, also known as p-hydroxyphenylpropionic acid or phloretic acid, is a sensitive fluorescent substrate of horseradish peroxidase, which is used as a pharmaceutical intermediate. At present, main production methods for the deaminotyrosine include using phenol, p-hydroxybenzaldehyde and p-methoxybenzaldehyde as raw materials for chemical synthesis. The chemical synthesis methods have the problems of a low yield, many by-products, environmental pollution and the like. Industrial products obtained by the chemical synthesis methods can be safely used for prevention and treatment of diseases after purification and detection. However, biotransformation is a more efficient and safer alternative method.

Phloretin, mainly distributed in the peel and root peel of juicy fruits such as apples and pears, can be ingested in daily diet. After entering intestines, the phloretin can be used by the intestinal flora and hydrolyzed to produce deaminotyrosine. Supplementation of probiotics with a phloretin hydrolyzing effect can increase the content of intestinal deaminotyrosine, so as to activate the immune response of the body and relieve the influenza virus infection.

In summary, it may be possible to develop probiotics that can activate the immune response of the body to relieve pathological characteristics of mice infected with influenza virus, so as to provide a safer and more efficient choice for prevention and relieving of influenza virus infection.

### SUMMARY

The first purpose of the present disclosure is to provide a strain of L. pentosus CCFM1227. The L. pentosus CCFM1227 has been collected in Guangdong Microbial Culture Collection Center on Mar. 21, 2022 with a collection number of GDMCC No: 62306, and the collection address is 5th floor, Building No. 59, Courtyard No. 100, Martyr Middle Road, Guangzhou City.

The second purpose of the present disclosure is to provide a probiotic preparation. The probiotic preparation contains the L. pentose CCFM1227.

In one embodiment of the present disclosure, the probiotic preparation contains wet bacteria or freeze-dried cells of the L. pentosus CCFM1227.

In one embodiment of the present disclosure, the probiotic preparation is a liquid bacterial agent or a solid bacterial agent.

In one embodiment of the present disclosure, a method for preparing the probiotic preparation includes: inoculating L. pentose CCFM1227 into a culture medium at inoculation amount of 2%-4% of the total mass of the culture medium, and performing culturing at 37° C. for 30 h to obtain a culture solution; subjecting the culture solution to centrifugation, and collecting bacteria; washing the bacteria with a phosphate buffer solution with a pH value of 7.2 for 3 times, and resuspending the bacteria with a freeze-drying protective agent to obtain a resuspension solution; and then subjecting the resuspension solution to freeze-drying by a vacuum freezing method to obtain a fermented agent of L. pentosus CCFM1227.

In one embodiment of the present disclosure, the probiotic preparation is a bacterial suspension of L. pentosus CCFM1227.

In one embodiment of the present disclosure, the bacterial suspension is a bacterial suspension prepared by mixing cells of L. pentose CCFM1227 with a freeze-drying protective agent.

In one embodiment of the present disclosure, the mass ratio of the freeze-drying protective agent to the bacteria is 2:1.

In one embodiment of the present disclosure, the freeze-drying protective agent includes skim milk powder, maltodextrin and sodium L-glutamate; and the ratio of the skim milk powder to the maltodextrin to the sodium L-glutamate is (8-10):(8-10): 1.

In one embodiment of the present disclosure, the culture medium is prepared by dissolving skim milk that is 10% of the total mass of the culture medium, 0.5% of glucose, 1.5% of tryptone and 0.3% of a yeast extract in water.

In one embodiment of the present disclosure, the culture medium has a pH value of 6.8.

The third purpose of the present disclosure is to provide a product. The product contains the L. pentose CCFM1227 or the probiotic preparation.

In one embodiment of the present disclosure, the product includes a drug or a food.

In one embodiment of the present disclosure, the viable count of L. pentosus CCFM1227 in the product is not less than 1×10⁶ CFU/mL.

In one embodiment, the drug contains the L. pentosus CCFM1227, a drug carrier and/or a pharmaceutical adjuvant.

In one embodiment, the drug carrier includes a microcapsule, a microsphere, a nanoparticle and a liposome.

In one embodiment, the pharmaceutical adjuvant includes an excipient and an additive.

In one embodiment, the pharmaceutical adjuvant includes an anti-adhesion agent, a penetration enhancer, a buffer, a plasticizer, a surfactant, a defoamer, a thickener, an encapsulation agent, an absorbent, a humectant, a solvent, a propellant, a solubilizer, a co-solvent, an emulsifier, a colorant, a pH regulator, a binder, a disintegrator, a filler, a lubricant, a wetting agent, an integrator, an osmotic pressure regulator, a stabilizer, a flow aid, a flavor modifier, a preservative, a foaming agent, a suspension aid, a coating material, an aromatic agent, a diluent, a flocculant, a deflocculant, a filter aid and a release blocker.

In one embodiment, the additive includes microcrystalline cellulose, hydroxypropyl methyl cellulose and refined lecithin.

In one embodiment, the dosage form of the drug includes a granule, a capsule, a tablet, a pill or an oral liquid.

In one embodiment, the food is a fermented food.

In one embodiment, the food is produced from the L. pentose CCFM1227 or the probiotic preparation.

In one embodiment, the food includes a dairy product, a bean product or a fruit or

vegetable product.

The fourth purpose of the present disclosure is to provide application of the L. pentose CCFM1227 or the probiotic preparation in preparation of a product having at least one of the following functions:
(1) relieving weight loss of mice infected with influenza virus;
(2) reducing lung damage of the mice infected with influenza virus;
(3) down-regulating the virus load of the lungs of the mice infected with influenza virus;
(4) down-regulating the level of IL-6 in the lungs and the serum of the mice infected with influenza virus;
(5) increasing the level of IFN-β in the lungs of the mice infected with influenza virus; and
(6) having the ability of fermenting phloretin to produce deaminotyrosine.

The fifth purpose of the present disclosure is to provide a method for relieving or treating influenza virus. The method includes administrating the product to a subject.

In one embodiment, the relieving or treating influenza virus includes, but is not limited to, at least one of the following effects:
(1) relieving weight loss;
(2) reducing lung damage;
(3) down-regulating the virus load of the lung;
(4) down-regulating the level of IL-6 in the lung and the serum; and
(5) increasing the level of IFN-β in the lung.

### Beneficial Effects

The present disclosure provides a strain of L. pentosus CCFM1227 capable of improving the innate immune response of mice infected with influenza virus so as to prevent and/or relive influenza virus infection, which is specifically embodied in that
(1) the weight loss of the mice infected with influenza virus is relieved;
(2) the total protein level of alveolar lavage fluid of the mice infected with influenza virus is reduced by 86%;
(3) the lung inflammation infiltration of the mice infected with influenza virus is reduced;
(4) the viral load of the lungs of the mice infected with influenza virus is reduced by 67%;
(5) the level of IL-6 in the lungs of the mice infected with influenza virus is reduced by 63%;
(6) the level of IL-6 in the serum of the mice infected with influenza virus is reduced by 21%;
(7) the level of IFN-β in the lungs of the mice infected with influenza virus is increased by 1 time; and
(8) the L. pentosus CCFM1227 is used for fermenting phloretin to produce deaminotyrosine.

Therefore, the L. pentosus CCFM1227 has a great application prospect in preparation of products (such as foods, drugs or health care foods) for preventing and/or relieving influenza virus infection.

### Biological Collection

A strain of L. pentosus CCFM1227 has been collected in Guangdong Microbial Culture Collection Center on Mar. 21, 2022 with a collection number of GDMCC No: 62306, and the collection address is 5th floor, Building No. 59, Courtyard No. 100, Martyr Middle Road, Guangzhou City.

### BRIEF DESCRIPTION OF FIGURES

FIG. 1 shows the weight level of experimental mice in different groups;

FIG. 2 shows the total protein level of alveolar lavage fluid of experimental mice in different groups;

FIG. 3 shows pathological sections of the lungs of experimental mice in different groups;

FIG. 4 shows the virus load of the lungs of experimental mice in different groups;

FIG. 5 shows the level of IL-6 in the lungs of experimental mice in different groups;

FIG. 6 shows the level of IL-6 in the serum of experimental mice in different groups;

FIG. 7 shows the level of IFN-β in the lungs of experimental mice in different groups; and

FIG. 8 shows the level of deaminotyrosine produced by fermentation of phloric acid using different strains.

### DETAILED DESCRIPTION

The present disclosure is further described below in combination with drawings attached to the specification and specific examples, but the examples are not intended to limit the present disclosure in any form. Unless otherwise specified, reagents, methods and apparatuses used in the present disclosure are conventional reagents, methods and apparatuses in the art.

SPF grade ICR female mice involved in the following examples were purchased from Beijing Vital River.

### Culture Media Involved in the Following Examples are as Follows.

A formulation of an MRS culture medium (1 L) includes: 10 g of peptone, 10 g of a beef extract, 5 g of yeast powder, 20 g of glucose, 2 g of K₂HPO₄, 2 g of diammonium citrate, 2 g of sodium acetate, 1 mL of Tween 80, 0.5 g of MgSO₄·7H₂O and 0.25 g of MnSO₄·4H₂O, and has a pH value of 7.2-7.4.

A formulation of an MRS solid culture medium (1 L) includes: 10 g of peptone, 10 g of a beef extract, 5 g of yeast powder, 20 g of glucose, 2 g of K₂HPO₄, 2 g of diammonium citrate, 2 g of sodium acetate, 1 mL of Tween 80, 0.5 g of MgSO₄·7H₂O, 0.25 g of MnSO₄·4H₂O and 20 g of agar, and has a pH value of 7.2-7.4.

A formulation of an M9 culture medium (1 L) includes: 0.2407 g of magnesium sulfate, 0.0110 g of calcium chloride, 2.9940 g of potassium dihydrogen phosphate, 4.7840 g of disodium hydrogen phosphate, 0.5001 g of ammonium chloride, 0.5000 g of sodium chloride and 0.5 mM of phloretin.

### Detection Methods Involved in the Following Examples are as Follows.

Detection method of viable count: National standard “GB 4789.35-2016 National Food Safety Standard, Food Microbiology Detection, Lactic Acid Bacteria Detection” is adopted.

Detection method of acidity: National standard GB 431334-2010 is adopted.

L. pentosus 1 is another strain isolated from different fecal samples by a same method.

### Example 1: Screening and Identification of Strains

### (1) Screening of Strains

After gradient dilution with sterile normal saline, fecal samples were coated on an MRS solid culture medium containing nystatin and cultured at 37° C. for 24 h. Bacterial colonies with different forms on the MRS solid culture medium were selected and isolated by streaking until pure single bacterial colonies with same forms were obtained. The pure bacterial colonies on the MRS solid culture medium were selected, inoculated into a 5 mL MRS culture medium and cultured at 37° C. for 12 h to obtain purified culture solutions.

### (2) Identification of Strains

The purified culture solutions obtained in step 1 were uniformly mixed and then centrifuged to remove a supernatant so as to obtain bacteria. The bacteria were sent to a company for genome sequencing, sequencing results were compared with sequences in an NCBI database, and strains No. CCFM1227 and SEQ ID No:1 were found to be L. pentosus.

### (3) Culture of Strains and Preparation of Bacterial Suspensions

The L. pentosus CCFM1227 and the L. pentosus 1 were inoculated into an MRS solid culture medium and cultured at 37° C. for 24 h, respectively. Bacterial colonies were observed, and bacteria were observed under a microscope. It was found that the bacterial colonies were milky white, round, convex and smooth, and the bacteria were campylobacter with slightly irregular shapes and round ends, which were usually present in single ones, pairs and small clusters.

The two strains of L. pentosus CCFM1227 and L. pentosus 1, were inoculated into an MRS liquid culture medium and cultured at 37° C. for 24 h, respectively, and then transferred into a fresh MRS liquid culture medium and cultured under the same conditions for 12 h. Bacteria were centrifuged at 8,000×g for 15 min, washed with 0.9% normal saline and centrifuged at 8,000×g again for 10 min. After a supernatant was discarded, the bacteria were collected, suspended with a 30% (m/v) sucrose solution and frozen and stored at −80° C. for later use.

When used for gavage of mice, the L. pentosus CCFM1227 and the L. pentosus 1 were taken out at-80° C., centrifuged to remove a supernatant and then resuspended with sterile normal saline to obtain bacterial suspensions for gavage.

### Example 2: Effect of L. pentosus CCFM1227 on Relieving Weight Loss of Mice Infected with Influenza Virus H1N1

SPF grade ICR female mice aged 3-4 weeks were divided into 4 groups including a normal group, a model group, a CCFM1227 experimental group and an L. pentosus 1 experimental group, with 8 mice in each group. The animals were raised with normal feed in the Experimental Animal Center of Yangzhou University, at a constant temperature of 21-26° C., a humidity of 40%-70%, a noise value of equal to or less than 60 dB and an animal illumination of 15-20 LX (all animal experimental procedures were reviewed and approved by the Animal Welfare and Ethics Management Committee of Yangzhou University).

An experiment was carried out in a period of 26 days in total, molding was performed on day 21, and the mice were infected with 10 μL of an H1N1 virus suspension with a concentration of 5LD₅₀ by nasal dropping after being anesthetized. During the experiment, the experimental groups were administrated with 0.2 mL of bacterial suspensions with a viable count of 1×10⁹ CFU/mL by gavage every day, while the normal group and the model group were only administrated with the same amount of sterile normal saline as a control, and all the groups were provided with free water and food. The mice were weighed every day after virus infection and then killed on day 27.

Grouping and treatment methods of the experimental animals are shown in Table 1.

TABLE 1
Grouping of experimental animals

| | Number/ | | |
|---|---|---|---|
| Group | group | Treatment method | |
| Normal | 8 | From day 1 to day 26, | |
| group | | 0.2 mL of sterile | |
| | | normal saline was | |
| | | administered by | |
| | | gavage every day | |
| Model | 8 | From day 1 to day 26, | On day 21, the mice |
| group | | 0.2 mL of sterile | were infected with 10 |
| | | normal saline was | μL of an H1N1 virus |
| | | administered by | suspension with a |
| | | gavage every day | concentration of |
| CCFM1227 | 8 | From day 1 to day 26, | 5LD₅₀ by nasal |
| experimental | | 0.2 mL of a bacterial | dropping |
| group | | suspension | |
| | | resuspended with | |
| | | sterile normal saline | |
| | | was administered by | |
| | | gavage every day | |
| L. pentosus 1 | 8 | From day 1 to day 26, | |
| experimental | | 0.2 mL of a bacterial | |
| group | | suspension | |
| | | resuspended with | |
| | | sterile normal saline | |
| | | was administered by | |
| | | gavage every day | |

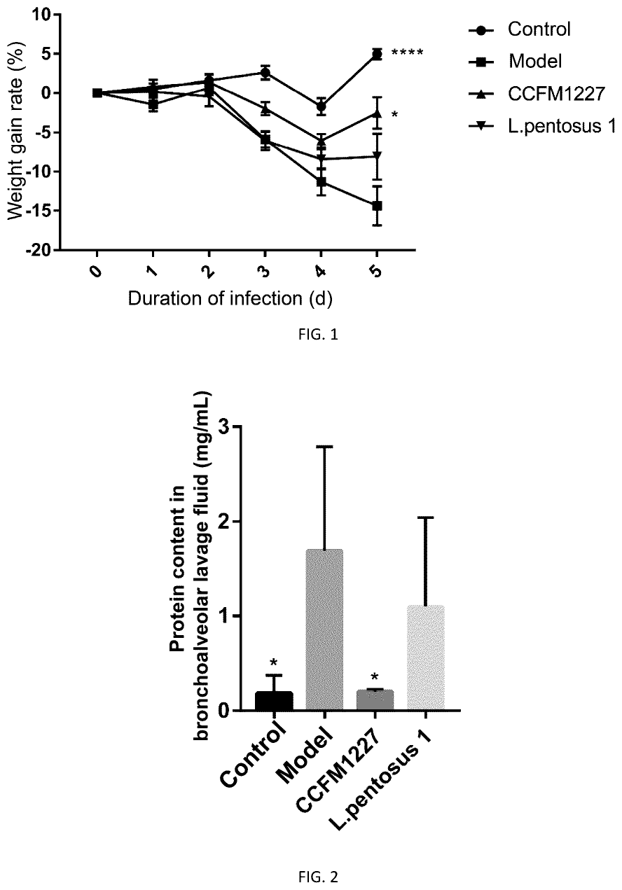

As can be seen from FIG. 1, the weight of the mice is obviously reduced during H1N1 virus infection, indicating that influenza virus infection leads to weight loss of the mice. In addition, compared with the model group and the L. pentosus 1 experimental group, the weight in the CCFM1227 experimental group is maintained basically stable, and the weight change rate is reduced by 11.8% and 5.54% compared with that in the model group and the L. pentosus 1 experimental group, respectively.

The above experimental results show that the L. pentosus CCFM1227 can relieve the weight loss caused by the influenza virus infection and has an obviously better effect than the L. pentosus 1.

### Example 3: Effect of L. pentosus CCFM1227 on Relieving Lung Damage of Mice Infected with Influenza Virus H1N1

Grouping and modeling methods of mice were the same as those in Example 2.

On day 21, the mice were killed to obtain alveolar lavage fluid.

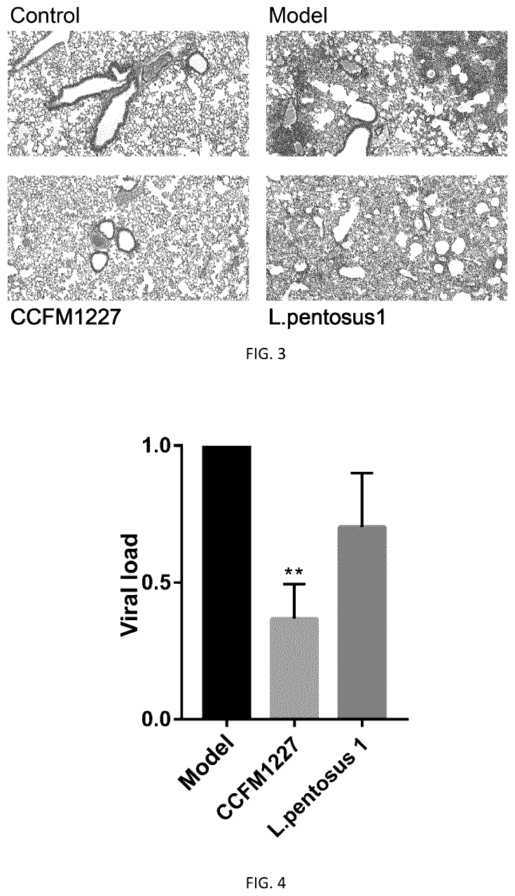

As can be seen from FIG. 2, the protein content of the alveolar lavage fluid of the mice in a normal group is small, while the protein content of the alveolar lavage fluid of the mice in a model group is obviously increased, indicating that infection with influenza virus H1N1 leads to lung damage of the mice and leads to infiltration of lung tissue fluid. In addition, compared with the model group, the protein content of the alveolar lavage fluid of the mice in a CCFM1227 experimental group is obviously reduced by 86% (P<0.01).

The above experimental results show that the L. pentosus CCFM1227 can relieve the lung damage caused by the influenza virus infection and has an obviously better effect than the L. pentosus 1.

### Example 4: Effect of L. pentosus CCFM1227 on Relieving Lung Inflammation of Mice Infected with Influenza Virus H1N1

Grouping and modeling methods of mice were the same as those in Example 2.

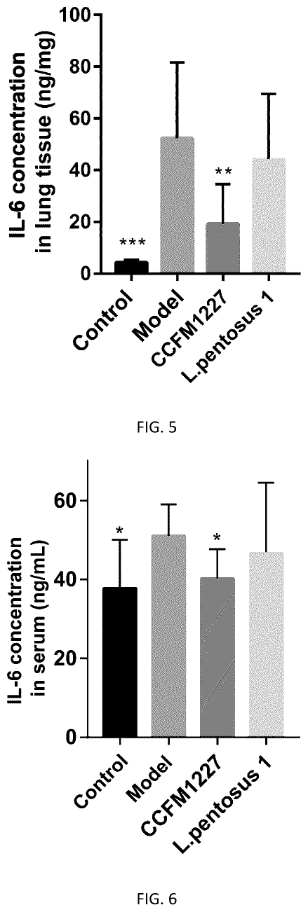

On day 21, the mice were killed, left lung lobes of the mice were taken to prepare pathological sections for histopathological analysis. Results are shown in FIG. 3.

As can be seen from FIG. 3, after infection with H1N1 influenza virus, the mice in a model group have serious lung inflammation infiltration, while the lung inflammation infiltration of the mice in experimental groups is obviously relieved, where the lung inflammation infiltration in the L. pentosus 1 experimental group is more serious than that in the CCFM1227 experimental group.

The above experimental results show that compared with the L. pentosus 1, the L. pentosus CCFM1227 is more effective in relieving the lung inflammation infiltration of the mice infected with influenza virus H1N1.

### Example 5: Effect of L. pentosus CCFM1227 on the Virus Load of the Lungs of Mice Infected with Influenza Virus H1N1

Grouping and modeling methods of mice were the same as those in Example 2.

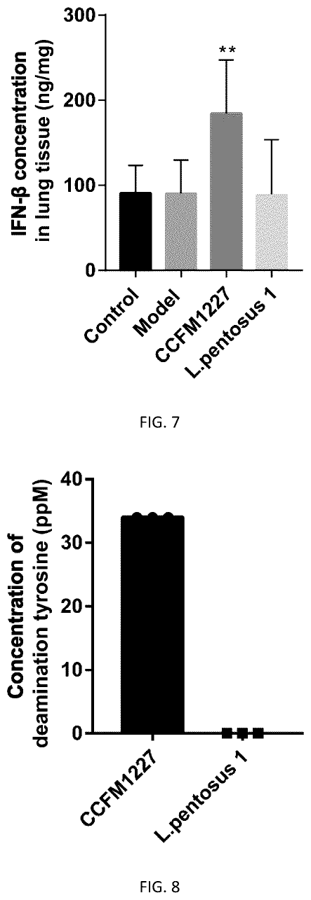

On day 21, the mice were killed to obtain lung tissues. After extraction of RNA, the virus load of the lung tissues was detected by qPCR. Results are shown in FIG. 4.

TABLE 2
Primers for qPCR detection

| Primers for | | |
|---|---|---|
| qPCR | | |
| detection | Sequence | |
| NP_F | GGCACCAAACGGTCTTACGA | SEQ ID NO: 2 |
| NP_R | TCACCTGATCAACTCCATTACCA | SEQ ID NO: 3 |

As can be seen from FIG. 4, the virus load of the lungs of the mice in an L. pentosus CCFM1227 group is obviously reduced by 67% compared with that in a model group (P<0.001), while L. pentosus 1 has little effect on the virus load of the lungs of the mice.

The above results show that the L. pentosus CCFM1227 can obviously reduce the virus load of the lungs of the mice.

### Example 6: Effect of L. pentosus CCFM1227 on the Level of an Inflammatory Factor IL-6 in the Lungs of Mice Infected with Influenza Virus H1N1

Grouping and modeling methods of mice were the same as those in Example 2.

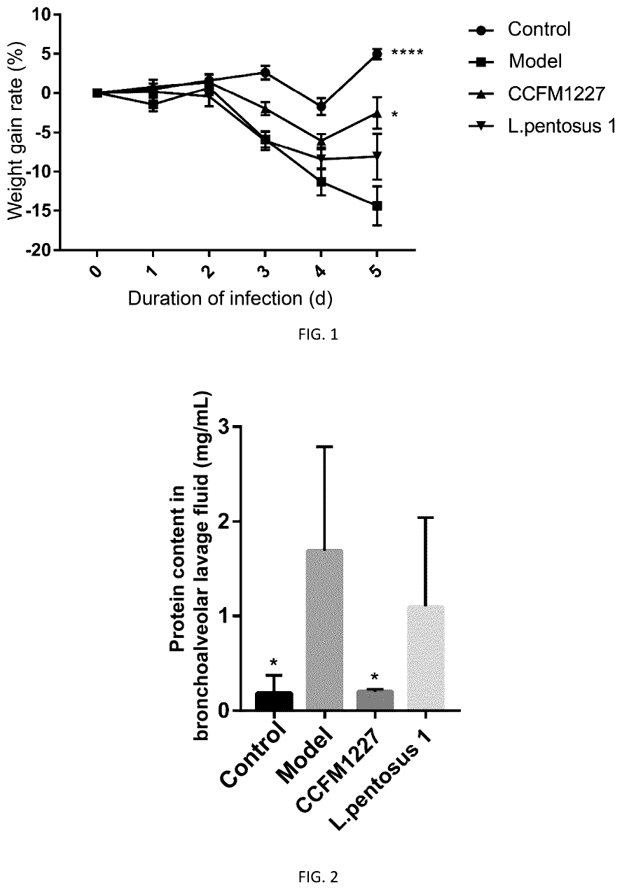

On day 21, the mice were killed to obtain lung tissues. The level of IL-6 in supernatants of the lung tissues was detected by ELISA. Results are shown in FIG. 5.

As can be seen from FIG. 5, the level of IL-6 in the lungs of the mice in a model group is obviously up-regulated compared with that in a normal group, the level of IL-6 in the lungs is obviously reduced after gavage of L. pentosus CCFM1227, and the level of IL-6 in the lungs of the mice in a CCFM1227 experimental group is obviously reduced by 63% compared with that in the model group (P<0.01).

The above experimental results show that the L. pentosus CCFM1227 has the effect of reducing the level of IL-6 in the lungs of the mice infected with influenza virus and has an obviously better effect than the L. pentosus 1.

### Example 7: Effect of L. pentosus CCFM1227 on the Level of an Inflammatory Factor IL-6 in the Serum of Mice Infected with Influenza Virus H1N1

Grouping and modeling methods of mice were the same as those in Example 2.

On day 21, the mice were killed, blood was centrifuged to obtain a supernatant, and the level of IL-6 in the serum was detected by an ELISA kit.

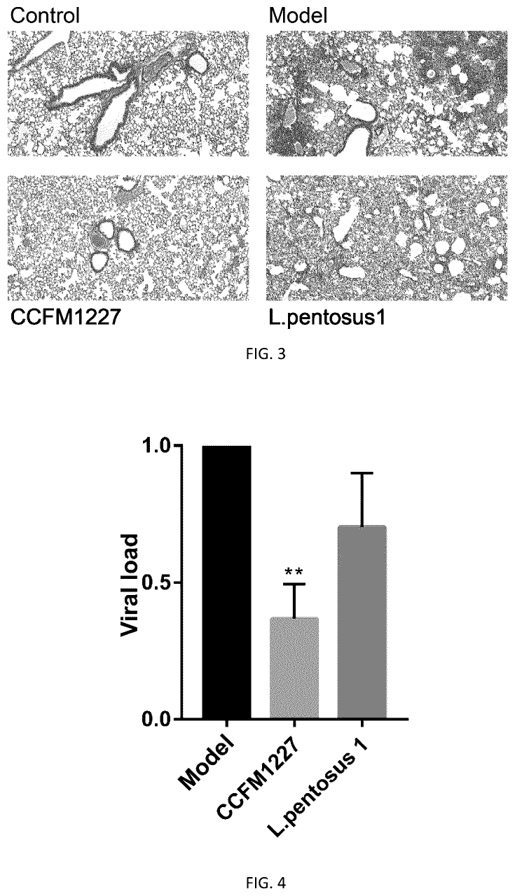

As can be seen from FIG. 6, the level of IL-6 in the serum of the mice in a model group is obviously increased compared with that in a normal group, and compared with the model group, the level of IL-6 in the serum of the mice in a CCFM1227 experimental group is obviously reduced by 21% (P<0.05).

The above experimental results show that the L. pentosus CCFM1227 can effectively reduce the level of an inflammatory factor IL-6 in the serum of the mice infected with influenza virus and further relieve inflammatory response caused by the influenza virus.

### Example 8: Effect of L. pentosus CCFM1227 on the Level of a Type I Interferon Cytokine IFN-β in the Lungs of Mice Infected with Influenza Virus H1N1

Grouping and modeling methods of mice were the same as those in Example 2.

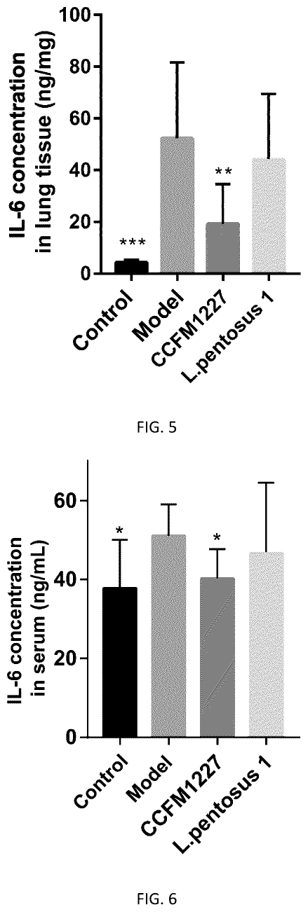

On day 21, the mice were killed to obtain lung tissues. The level of IFN-β in supernatants of the lung tissues was detected by ELISA. Results are shown in FIG. 7.

As can be seen from FIG. 7, the level of IFN-β in the lungs of the mice in a model group is not obviously changed compared with that in a normal group, while the level of IFN-β in the lungs of the mice in a CCFM1227 experimental group is obviously increased by 1 time compared with that in the model group (P<0.01), indicating that L. pentosus 1 has no effect on the level of IFN-β in the lungs.

The above experimental results show that the L. pentosus CCFM1227 has the effect of improving the antiviral pathway response of an innate immunity type I interferon of the mice infected with influenza virus, helps to improve the antiviral ability of the body and has an obviously better effect than the L. pentosus 1.

### Example 9: Effect of L. pentosus CCFM1227 on Fermenting Phloretin

L. pentosus CCFM1227 and L. pentosus 1 were subjected to streak culture in an MRS culture medium and activated by liquid culture for three generations, respectively. 200 μl of a bacterial solution was added into a 5 mL MRS culture medium, subjected to anaerobic culture at 37° C. to an early plateau phase, centrifuged at 3,400×g at 4° C. for 20 min to remove the MRS culture medium and then washed once with PBS to obtain bacterial sludge.

1 mL of an M9 culture medium (g/L) containing 0.5 mM phloretin was added into the bacterial sludge and subjected to anaerobic culture at 37° C. for 24 h. After completion of resting culture of microorganisms, centrifugation was performed at 7,000×g at 4° C. for 10 min, 1 ml of a supernatant was added into a 1.5 mL centrifuge tube and subjected to vortex oscillation for 10 s, and then, 100 μL of the supernatant was added into a new 1.5 mL centrifuge tube. 800 μL of precooled methanol was added, shaken and mixed uniformly. A resulting mixture was subjected to protein settlement on ice for 30 min and then centrifuged at 15,000×g at 4° C. for 10 min to obtain a supernatant. The supernatant was concentrated under vacuum at 45° C. for 2-4 h, resuspended in 100 μl of water containing 20% of methanol, centrifuged at 15,000×g at 4° C. for 10 min and then filtered with a 0.2 μm filter membrane. The concentration of deaminotyrosine was detected by liquid chromatography-mass spectrometry.

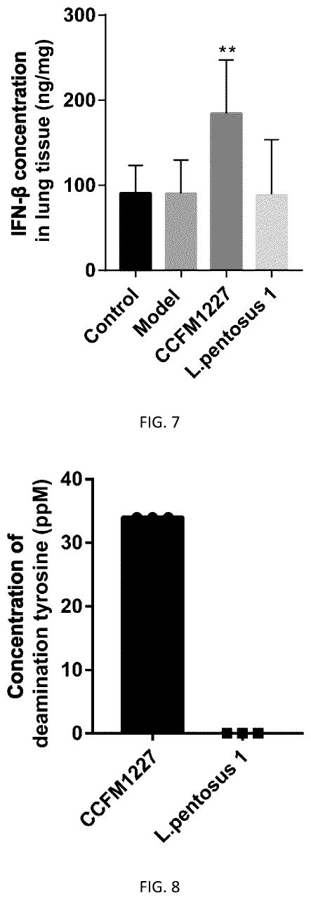

As can be seen from FIG. 8, the L. pentosus CCFM1227 can be used for fermenting phloretin to produce deaminotyrosine, while the L. pentosus 1 cannot be used for producing deaminotyrosine.

### Example 10: Preparation of a Bacterial Powder Containing L. pentosus CCFM1227

A seed liquid of L. pentosus CCFM1227 collected in a bacterial collection tube was inoculated into an MRS culture medium at an inoculation amount of 3% of the total mass of the culture medium, and cultured at 37° C. for 12 h to obtain a culture solution. The culture solution was centrifuged, and bacteria were collected. The bacteria were washed with a phosphate buffer solution with a pH value of 7.2 for 3 times, and then resuspended with a trehalose freeze-drying protective agent with a trehalose concentration of 100 g/L to obtain a resuspension solution, where the mass ratio of the freeze-drying protective agent to the bacteria was controlled at 2:1. Then, the resuspension solution was precooled at −80° C. for 1.5 h, and immediately transferred to a freeze dryer for drying for 24 h to obtain a bacterial powder containing L. pentosus CCFM1227.

### Example 11: Preparation of a Yogurt Containing L. pentosus CCFM1227

Milk powder, inulin, stevia sugar and water were mixed at a weight ratio of 20:5:5:75 and then homogenized to prepare a fermentation raw material. The fermentation raw material was sterilized at an ultrahigh temperature of 121° C. for 300 s, cooled to 42° C., inoculated into a mixed bacterial powder of Lactobacillus bulgaricus and Streptococcus thermophilus, and then fermented at 42° C. for 12 h, where the bacteria concentration of the L. bulgaricus and the S. thermophilus was controlled to 105 CFU/g and 107 CFU/g, respectively for preparation. A fermented product was cooled to 37° C., and a freeze-dried bacterial powder containing L. pentosus CCFM1227 was added, where the added amount of the freeze-dried bacterial powder containing L. pentosus CCFM1227 was 10⁹ CFU L. pentosus CCFM1227/mL yogurt. Then, a resulting mixture was stirred, canned and stored at 4° C. for 2 days to complete natural after-ripening so as to prepare a probiotic yogurt.

Although the present disclosure has been disclosed as above through the preferred examples, the examples are not intended to limit the present disclosure. For anyone familiar with the art, various changes and modifications can be made without departing from the spirit and scope of the present disclosure. Therefore, the protection scope of the present disclosure should be as defined in the claims.

What is claimed is:

1. A product comprising Lactobacillus pentosus (L. pentosus) CCFM1227, wherein the L. pentosus CCFM1227 has been deposited in Guangdong Microbial Culture Collection Center on Mar. 21, 2022 with a deposit number of GDMCC No: 62306.

2. The product according to claim 1, wherein the product comprises a drug or a food.

3. The product according to claim 1, wherein the viable count of L. pentosus CCFM1227 in the product is not less than 1×10⁶ CFU/mL.

4. The product according to claim 2, wherein the drug contains L. pentosus CCFM1227, a drug carrier and/or a pharmaceutical adjuvant.

5. The product according to claim 2, wherein the food is a fermented food.

6. The product according to claim 5, wherein the food comprises a dairy product, a bean product or a fruit or vegetable product.

7. A method for relieving or treating influenza virus, comprising administrating the product according to claim 1 to a subject.

8. The method according to claim 7, wherein the relieving or treating influenza virus comprises at least one of the following effects:
(1) relieving weight loss;
(2) reducing lung damage;
(3) down-regulating the virus load of the lung;
(4) down-regulating the level of IL-6 in the lung and the serum; and
(5) increasing the level of IFN-β in the lung.


SEQUENCE LISTING (3 sequences)

SEQ ID NO: 1 - DNA, 963 bp, Lactobacillus pentosus
  source 1..963: mol_type = genomic DNA
nnngngggnngggtgctatacatgcagtcgaacgaactctggtattgattggtgcttgcatcatgatttacatttgagtgagtggcgaactggtgagtaacacgtgggaaacctgcccagaagcgggggataacacctggaaacagatgctaataccgcataacaacttggaccgcatggtccgagtttgaaagatggcttcggctatcacttttggatggtcccgcggcgtattagctagatggtggggtaacggctcaccatggcaatgatacgtagccgacctgagagggtaatcggccacattgggactgagacacggcccaaactcctacgggaggcagcagtagggaatcttccacaatggacgaaagtctgatggagcaacgccgcgtgagtgaagaagggtttcggctcgtaaaactctgttgttaaagaagaacatatctgagagtaactgttcaggtattgacggtatttaaccagaaagccacggctaactacgtgccagcagccgcggtaatacgtaggtggcaagcgttgtccggatttattgggcgtaaagcgagcgcaggcggttttttaagtctgatgtgaaagccttcggctcaaccgaagaagtgcatcggaaactgggaaacttgagtgcagaagaggacagtggaactccatgtgtagcggtgaaatgcgtagatatatggaagaacaccagtggcgaaggcggctgtctggtctgtaactgacgctgaggctcgaaagtatgggtagcaaacaggattagataccctggtagtccataccgtaaacgatgaatgctaagtgtttggagggtttccgcccttcagtgctgcagctaacgcattaagcattccgcctggggagtacggccgcaaggctgaaactcaaggaattgacgggggcccgcacaagcggtggagcatgtggtttattcgaagctacgcg

SEQ ID NO: 2 - DNA, 20 bp, synthetic construct
  source 1..20: mol_type = other DNA
ggcaccaaacggtcttacga

SEQ ID NO: 3 - DNA, 23 bp, synthetic construct
  source 1..23: mol_type = other DNA
tcacctgatcaactccattacca